(19) United States
(12) Patent Application Publication
(10) Pub. No.: US 2019/0000662 A1
(43) Pub. Date: Jan. 3, 2019

(54) ADJUSTABLE SLEEP APNEA ORAL APPLIANCE

(71) Applicant: SELANE PRODUCTS, INC., Chatsworth, CA (US)

(72) Inventor: Rob Veis, Chatsworth, CA (US)

(21) Appl. No.: 16/060,403

(22) PCT Filed: Dec. 12, 2016
(86) PCT No.: PCT/US2016/066240

§ 371 Date: Jun. 7, 2018

Related U.S. Application Data
(60) Provisional application No. 62/265,963, filed on Dec. 10, 2015.

(51) Int. Cl. A61F 5/56; A61C 7/08

(52) U.S. Cl. CPC A61F 5/566; A61C 7/36; A61C 7/08

(57) ABSTRACT

An oral appliance for treating sleep apnea while repositioning the teeth of a subject including an upper dental tray and a lower dental tray, the upper dental tray having a downwardly extending projection in an anterior portion of the upper dental tray. The distal end of the projection extends below the incisal surface of the lower dental tray such that the anterior surface of the projection is adjacent to an anterior lingual surface of the lower dental tray when the appliance is worn by the subject, thereby limiting the anterior movement of the upper dental tray with respect to the lower dental tray and alleviating snoring and/or apnea.

### CROSS-REFERENCE TO RELATED APPLICATIONS

This application claims the benefit of priority from U.S. Patent Application No. 62/265,963, filed on Dec. 10, 2015 and titled ADJUSTABLE SLEEP APNEA ORAL APPLIANCE.

### BACKGROUND

Sleep apnea is a disorder characterized by abnormal pauses in breathing or instances of abnormally low breathing during sleep. Each pause in breathing, called an apnea, can last from a few seconds to minutes (typically lasting 20 to 40 seconds) and may occur 5 to 30 times or more an hour. Sleep apnea results from a partial-to-complete blockage of a subject's airway. Increased air speed through the airway causes an increase in dynamic pressure and a corresponding drop in static pressure. The decreased static pressure can in some instances draw back the lower jaw and tongue and thereby block the airway. This blockage can increase to the point of becoming complete, which at least temporarily interrupts breathing.

Subjects are generally at greater risk for sleep apnea if they are overweight or have conditions such as diabetes, hypertension, or chronic nasal congestion. There are a variety of factors, however, which can lead to sleep apnea. One factor is the presence of a narrow maxilla and/or mandible in a subject. Maxillary constriction may increase nasal resistance and alter the tongue posture, leading to narrowing of the retroglossal airway. Constriction of the maxilla and/or the mandible generally reduces intraoral air volume and tends to force the tongue back into the posterior airway space, leading to obstructive sleep apnea during sleep.

Orthodontics is a field of dentistry which focuses on the repositioning of a subject's teeth and jaws for aesthetic or other reasons, for example due to the “overcrowding” of a subject's teeth. Orthodontic methods typically require a subject to make continuous use of a dental appliance for a period of time in order to achieve results. The use of such appliances precludes the concurrent use of currently available oral appliances for treating sleep apnea. There remains a need therefore for improved devices and methods for treating sleep apnea in users of orthodontic appliances who experience sleep apnea.

### SUMMARY

The present invention is an oral appliance 10 for use in treating snoring and/or sleep apnea in a subject which includes dental trays 15 and preferably also orthodontic trays 25. The appliance has an upper dental tray 100 and a lower dental tray 200, each of which has interior surface 11, an exterior surface 13, a right side 12, a left side 14, an anterior portion 16, a posterior portion 18, a coronal surface 30, a buccal wall 32, and a lingual wall 34. The upper and lower dental trays each also comprise a receptacle 20 in the interior surface 11, the receptacle having a bottom surface 21, a buccal wall 22, and a lingual wall 24. In addition, the upper dental tray 100 includes an incisal surface 101 and the lower dental tray 200 includes an incisal surface 201 in the anterior portion of each of these trays, and these incisal surfaces come into contact when the appliance is worn by the subject. The upper dental tray 100 further includes a downwardly extending projection 400 having a proximal end 402, a distal end 404, an anterior surface 401, and a posterior surface 403, where the projection 400 is connected to and extends downwardly from the upper dental tray 100 in an anterior portion of the upper dental tray 100. The distal end 404 of the projection 400 extends below the incisal surface 201 of the lower dental tray 200 such that the anterior surface of the projection 400 contacts or is adjacent to an anterior lingual surface 204 of the lingual wall 24 of the lower dental tray 200 when the appliance is worn by the subject, thereby limiting the anterior movement of the upper dental tray 100 with respect to the lower dental tray 200 and alleviating snoring and/or apnea when the oral appliance is used by the subject.

The receptacles 20 of the upper dental tray 100 and the lower dental tray 200 can each be configured to receive and retain an orthodontic tray 25, or alternatively the receptacles 20 of the upper dental tray 100 and the lower dental tray 200 can each be configured to reposition one or more teeth of a subject and/or to change the configuration of a subject's mandible and/or maxilla when the appliance is worn by the subject. When the present appliance system includes orthodontic trays, each of the orthodontic trays 25 can be received within respective receptacles 20 of the upper dental tray 100 and the lower dental tray 200 of the oral appliance such that the outer surface (313 or 413) of a respective orthodontic tray contacts the interior surface 11 of a tray receptacle 20 of a respective dental tray 15. In this embodiment, a series of orthodontic trays 25 is preferably used, and each of the orthodontic trays in the series comprises receptacles having different configurations in order to change the position of a subject's teeth and/or the shape of the subject's jaw. Preferably, the receptacles 20 of the upper dental tray 100 and the lower dental tray 200 are shaped to receive all of the orthodontic trays 25, advantageously in a reversibly securable manner.

In one embodiment, the downwardly extending projection 400 is attached to a base 420 to form an insert 410. The base 420 has an upper surface 421, a lower surface 423, an anterior side 426, a posterior side 428, a right side 422, and a left side 424. The insert 410 is configured to be retained by an insert receptacle 103, which receives the base portion 420 of the insert and reversibly secures the insert 410 to the upper dental tray 100, preferably through an interference fit. The insert receptacle preferably has anterior opening 116, a lower surface 111, a right side wall 112, a left side wall 114, and a posterior wall 118, and the base 420 preferably slides horizontally through the anterior opening such that the right side, left side, and rear side of the base 420 contact the right side, left side, and rear side of the insert receptacle 103 when the insert 410 is positioned in the insert receptacle. A plurality of inserts can be provided such that the projections 400 of each of the inserts is positioned on the base 420 of the respective insert in a relatively more anterior or posterior position with respect to the projection of the other inserts.

### FIGURES

FIG. 1 is an exploded, left side, perspective view of an embodiment of the present appliance.

FIG. 2 is a left side elevation of the appliance of FIG. 1.

FIG. 3 is a right side, top perspective view of the lower portion of the appliance of FIG. 1.

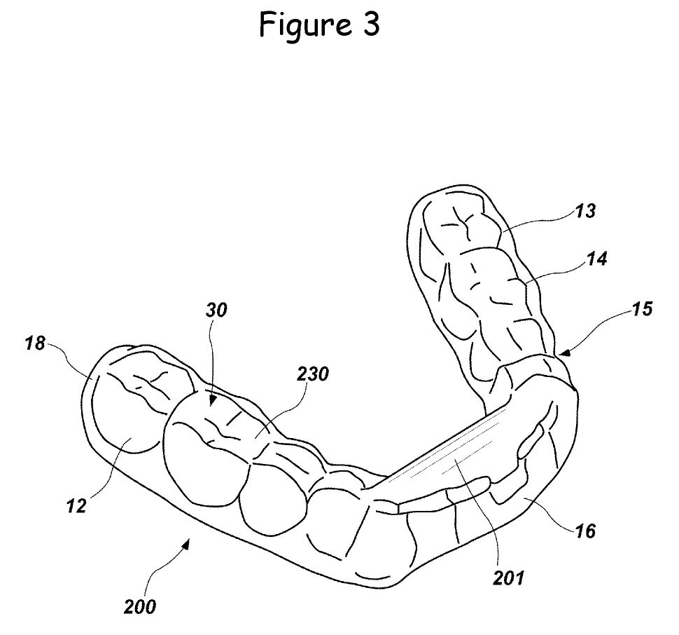

FIG. 4 is a left side, bottom perspective view of the lower portion of the appliance of FIG. 1.

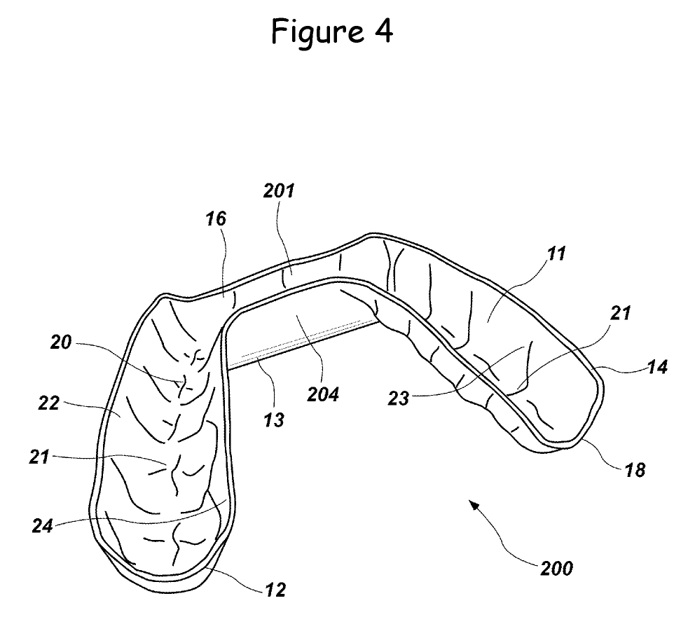

FIG. 5 is a right side, top perspective view of the upper portion of the appliance of FIG. 1.

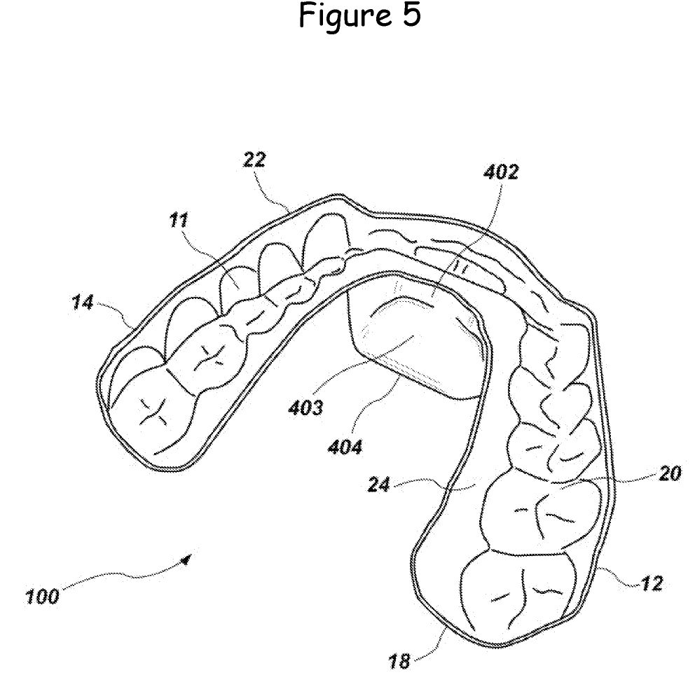

FIG. 6 is a bottom perspective view of the lower upper of the appliance of FIG. 1.

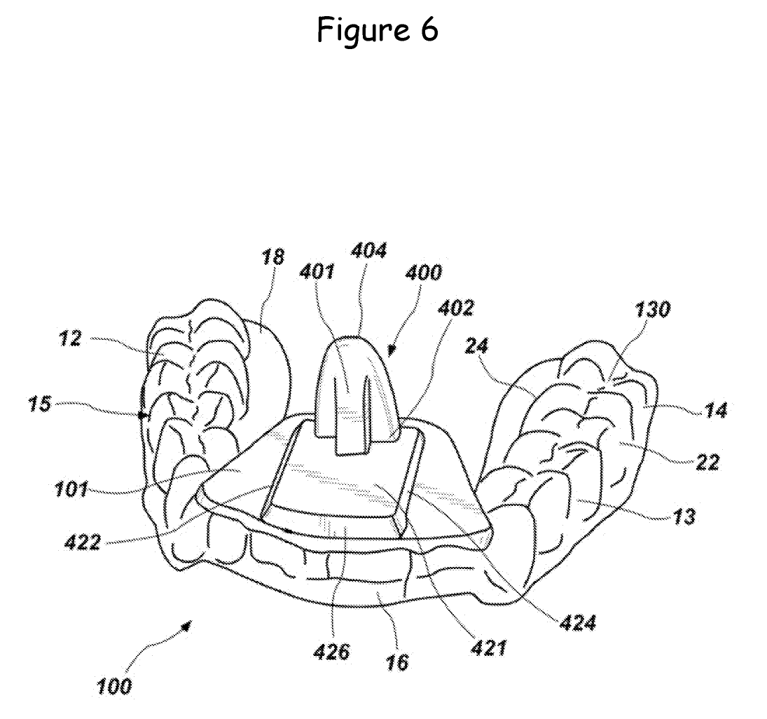

FIG. 7 is a left side, bottom perspective view of the upper portion of an embodiment of the present appliance showing an insert.

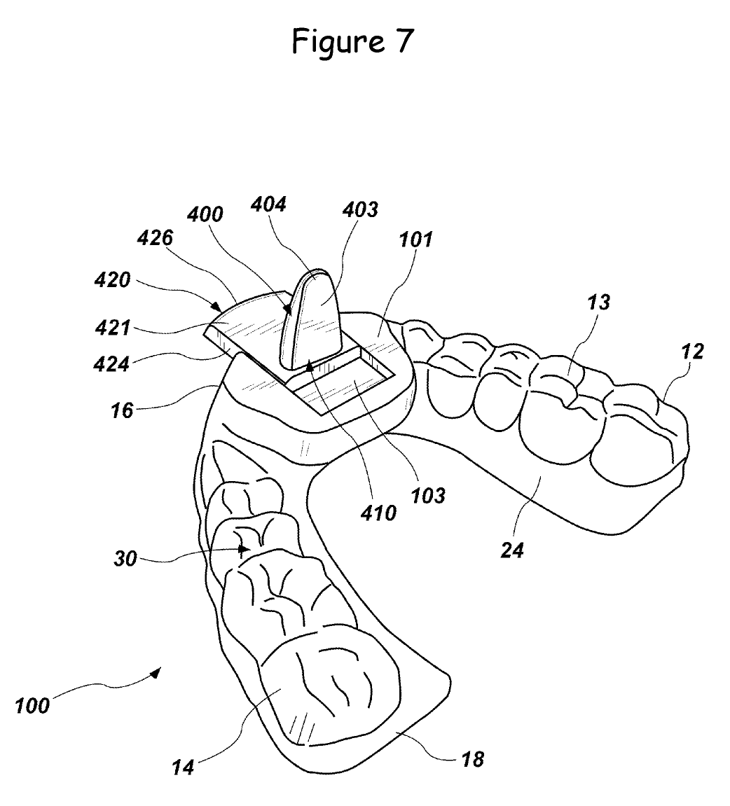

FIG. 8 is a bottom perspective view of the upper portion of the appliance of FIG. 7, showing the insert removed.

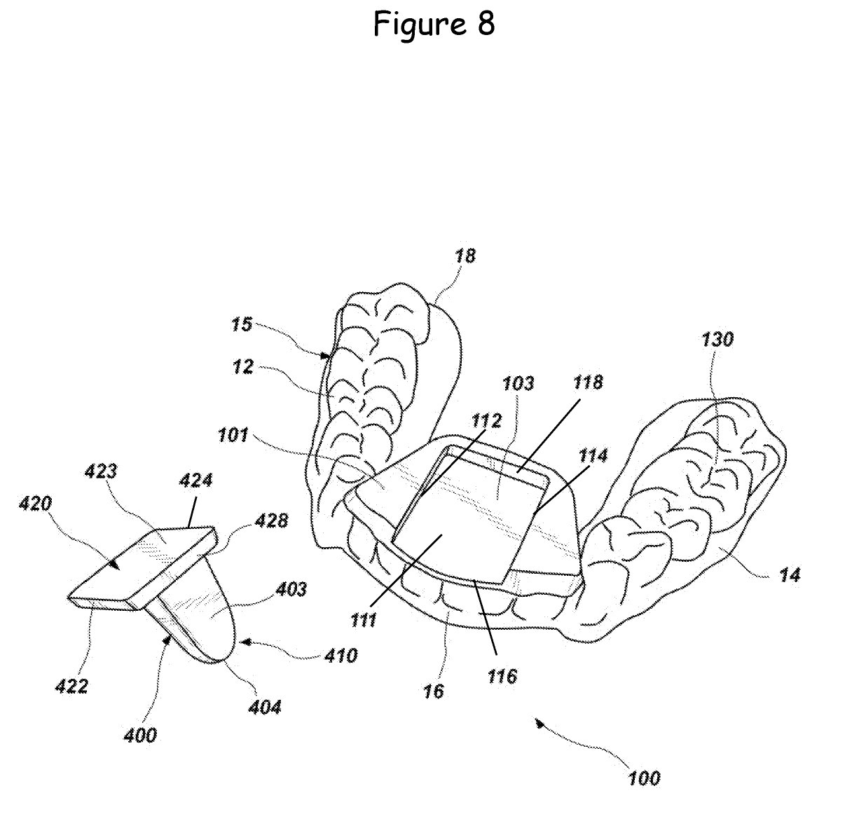

FIG. 9 is a bottom perspective view of the upper portion of the appliance of FIG. 7, showing a plurality of inserts.

FIG. 10 is an exploded, left side, perspective view of an embodiment of the present appliance of FIG. 1, additionally showing orthodontic trays for use with the present appliance.

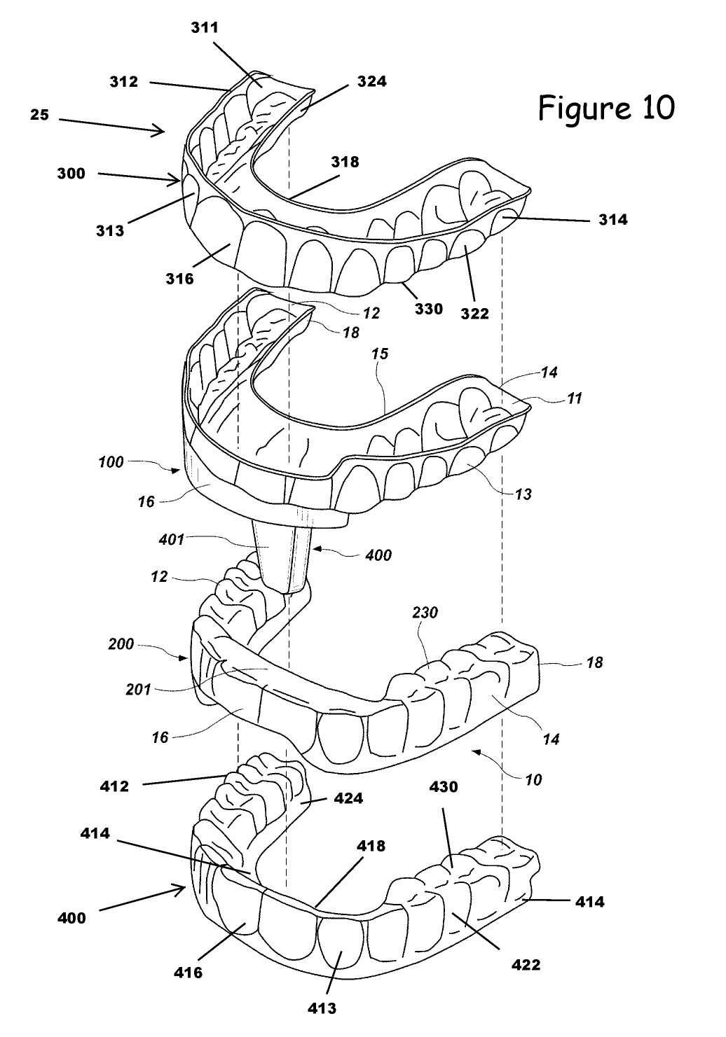

### DESCRIPTION

### Definitions

As used herein, the following terms and variations thereof have the meanings given below, unless a different meaning is clearly intended by the context in which such term is used.

“Above” refers to the relative position of the present appliance or a component thereof which is closer to or is toward an upper portion of a subject's body when the appliance is being used, while “below” refers to the opposite relative position of the present appliance or a component thereof, i.e. being closer to or in the direction of a lower portion of a subject's body when the appliance is being used

“Anterior” means in the direction of or toward or adjacent the front portion (opening) of a subject's mouth.

“Apnea” and “sleep apnea” refer to a temporary cessation of breathing and/or to instances of shallow or infrequent breathing during sleep, generally caused by a blockage of a subject's airway (referred to as obstructive sleep apnea).

“Axial plane” refers to an imaginary plane that divides the body into cranial and caudal (upper and lower) portions.

“Buccal” means in the direction of or toward a subject's cheek. In relation to a subject's teeth, this refers to the side of the teeth facing the cheek.

“Coronal plane” refers to a hypothetical planar surface that extends through the body from the head to the feet, and divides the body into front and rear halves.

“Coronal surface” is the biting surface of a tooth or of the portion of the present appliance which is adjacent the coronal surface of a tooth. In the posterior portion of a jaw or of the present appliance, the coronal surface can be referred to as an “occlusal surface,” and the coronal surface in an anterior portion of a jaw or of the present appliance can be referred to as an “incisal surface.” “Coronal surface” may thus refer to the corresponding surface of a dental tray which contacts the other dental tray when the present device is worn by a user, i.e. to a lower surface of an upper dental tray or to an upper surface of a lower dental tray.

“Dental tray” refers to a structure comprising a receptacle for receiving the teeth of a subject. In some embodiments, the receptacle has an opening for receiving teeth and an interior surface which contacts the subject's teeth directly. In other embodiments, the receptacle receives an orthodontic tray.

“Downward” and “downwardly” mean in the direction of or toward a lower portion of a subject's body. “Upward” and “upwardly” mean in the opposite direction, i.e. in the direction of or toward an upper portion of a subject's body.

“Elongated” refers to a configuration or shape having a length which is longer than its width.

“Horizontal,” with respect to the present appliance, refers to disposition in a plane approximately perpendicular to the sagittal and/or the coronal plane of a subject, i.e. within 15 degrees of such a perpendicular plane.

“Insert” refers to a component of the present appliance which can be secured, preferably reversibly secured, to another component of the appliance which retains the insert portion.

“Labial” means in the direction of, toward, or adjacent to a subject's lips. In relation to a subject's teeth, this refers to the side of the front teeth facing the lips.

“Lateral” means away from the sagittal plane of a subject.

“Left” means to the left of the center sagittal plane of a subject, from the perspective of the subject.

“Lingual” means in the direction of, toward, or adjacent to a subject's tongue. In relation to a subject's teeth, this refers to the side of the teeth facing the tongue.

“Lower” refers to the relative position of a component in the present appliance which is closer to or toward a lower portion of a subject's body when the component is being used.

“Mandibular” refers to the lower jaw.

“Mandibular dentition” refers to the teeth of the lower jaw.

“Maxillary” refers to the upper jaw.

“Maxillary dentition” refers to the teeth of the upper jaw.

“Mechanically connected” means physically connected, either through a connection based on direct physical contact or via another intermediate mechanical structure.

“Medial” means toward the center sagittal plane of a subject.

“Orthodontic” refers to a feature or component of an appliance, or an appliance itself, which repositions the teeth and/or jaw(s) of a subject.

“Orthodontic tray” refers to a dental tray for receiving the upper or lower dentition of a subject. An interior surface of an orthodontic tray contacts the subject's teeth directly with sockets or depressions sized to receive a subject's teeth.

“Posterior” means in the direction of or toward or adjacent the rear portion of a subject's mouth.

“Right” means to the right of the center sagittal plane of a subject, from the perspective of the subject.

“Sagittal plane” refers to an imaginary plane that travels vertically from the top to the bottom of the body of a subject, dividing it into left and right portions.

“Subject” refers to a user of the present appliance, usually a human user.

“Thermoplastic” refers to a material, generally a polymer material, which may be softened by heat and hardened by cooling in a reversible physical process. The thermoplastic materials used in some components of the present appliance retain their shape at 100° F. and preferably become soft (deformable) at a temperature of 212° F. or below.

“Tray” and “dental tray,” as used herein, refer to a generally U-shaped portion of the present appliance comprising an open area for receiving the maxillary or mandibular teeth of a subject, as the case may be.

“Upper” refers to the relative position of a component in the present appliance which is closer to or toward an upper portion of a subject's body when the component is being used.

“Vertical,” with respect to the present appliance, refers to disposition in a plane approximately parallel to the sagittal and/or the coronal plane of a subject, i.e. within 15 degrees of such a parallel plane. Preferably, vertical refers to a direction toward or away from a subject's head or feet.

The term “comprise” and variations of the term, such as “comprising” and “comprises,” are not intended to exclude other additives, components, integers or steps. The terms “a,” “an,” and “the” and similar referents used herein are to be construed to cover both the singular and the plural unless their usage in context indicates otherwise.

### Oral Appliance

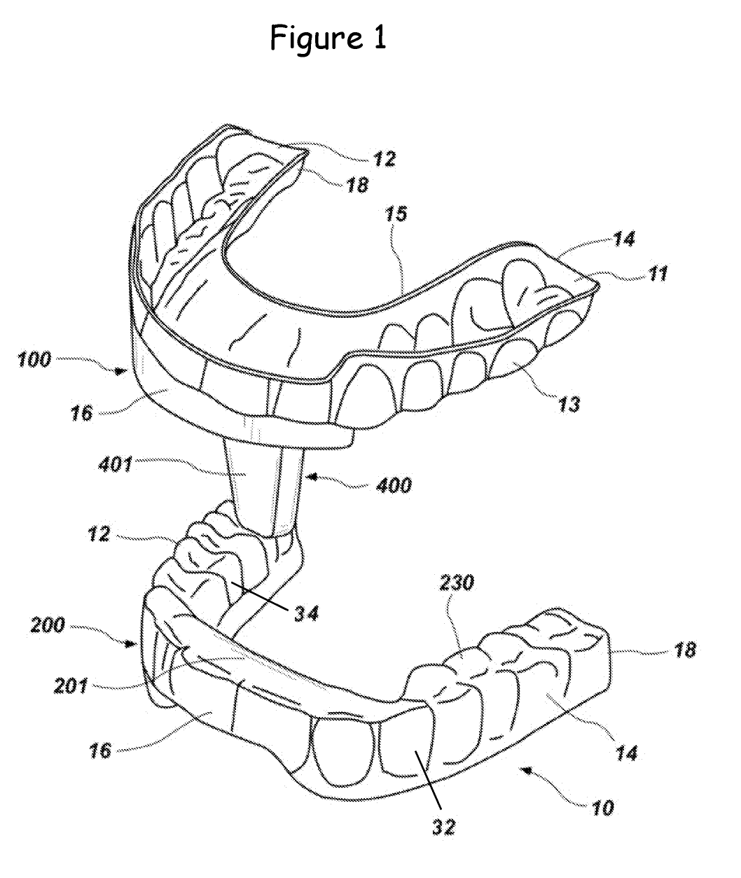

FIG. 1 illustrates an embodiment of the present sleep apnea oral appliance. The present appliance 10 generally comprises a pair of dental trays 15, an upper tray 100 and a lower tray 200, which cooperate to position a subject's jaws so as to treat or avoid sleep apnea and/or snoring. The dental trays 15 are worn during sleep, usually at night. The upper tray 100 is fitted onto a subject's maxillary dentition, while the lower tray 200 is fitted over the subject's mandibular dentition. The tray portions 15 of the present device have an interior surface 11, an exterior surface 13, a right side 12, a left side 14, an anterior portion 16, a posterior portion 18, a buccal wall 32 and a lingual wall 34, and each comprise a generally U-shaped tray receptacle 20 formed on one horizontal side of the tray 15 to fit over a subject's dentition.

In one embodiment, the tray receptacle 20 is configured to receive the teeth of a subject and to contact the subject's teeth on the interior surface 11 of the tray 15. The receptacle 20 comprises lateral contiguous walls 23 which extend from a bottom surface 21 facing the coronal surfaces of a subject's teeth toward the maxilla or mandible, respectively, i.e. buccal wall 22 and lingual wall 24, so as to cover some or all of the buccal and lingual sides of some or all of a subject's teeth. The trays 15 can be formed to conform to a subject's pre-existing dentition, or in a preferred embodiment can be formed to accomplish a change in the configuration of a subject's existing dentition and/or in the shape of a subject's mandible and/or maxilla, as described further below.

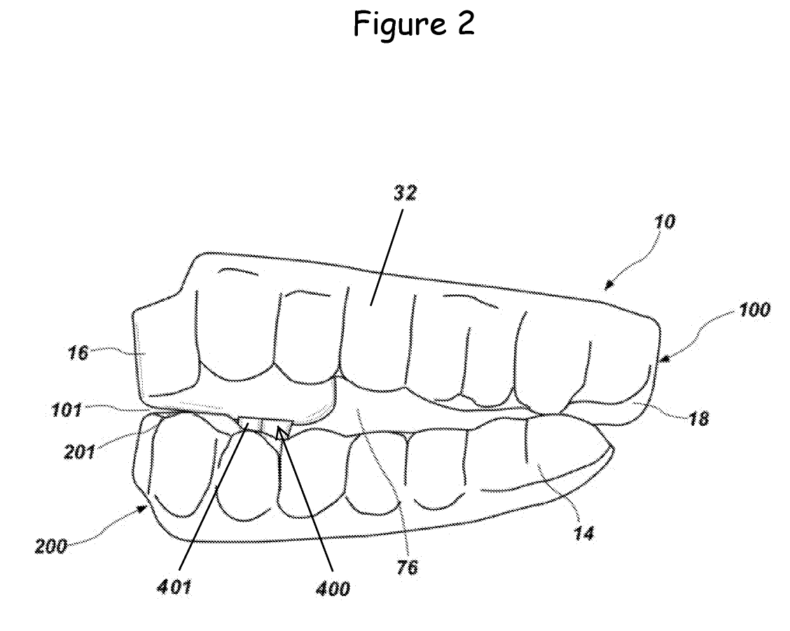

The exterior portion of each of the dental trays 15 comprises a coronal surface 30 formed on the horizontal side of the tray opposite the tray receptacle 20, i.e. on the exterior surface 13 of the tray 15. The coronal surface 130 of the upper tray 100 preferably contacts the coronal surface 230 of the lower tray 200 in the posterior portion 18 of the right 12 and left 14 sides of the appliance 10, approximately in the molar area of a user's dentition (as shown in FIG. 2), but there can also be contact elsewhere on the coronal surfaces of the upper tray 100 and lower tray 200.

Preferably, when the present appliance is in use, at least a portion of an anterior incisal surface 101 of the upper tray 100 is in contact with an anterior incisal surface 201 of the lower tray 200. When posterior coronal surfaces of the upper tray 100 and lower tray 200 are in contact and the anterior incisal surfaces 101 and 201 are also in contact, the appliance 10 will be stably positioned within the mouth of a user. In order to allow a user to breathe through the mouth, if necessary, one or more openings 76 between the upper tray 100 and 200 can be provided. The openings 76 shown in FIG. 2 are positioned posteriorly with respect to the upper incisal surface 101 and the lower incisal surface 201, though anterior openings are also possible.

Preferably, the upper incisal surface 101 extends further downwardly than the lower (coronal) surface of the appliance in the region of the teeth behind the front teeth, and/or the lower incisal surface 201 extends further upwardly than the upper (coronal) surface of the appliance in the region of the teeth behind the front teeth. In this way, the user's mouth is maintained in a relatively open position. Maintaining a subject's mouth in a slightly open position by separating the upper and lower jaws in this way also helps to treat apnea.

In the illustrated embodiments, the incisal surface 101 of the upper tray 100 comprises a downwardly extending projection 400, which has a proximal end 402, a distal end 404, an anterior surface 401, and a posterior surface 403. The projection 400 extends distally from the incisal surface 101 of the upper tray 100, i.e. downwardly when worn by a subject wearing the present device 10. The distal end 404 of the projection 400 extends downwardly beyond the incisal surface 201 of the lower tray 200 when worn by a subject, such that a portion of the distal end 404 of the projection 400 is positioned lingually with respect to an exterior surface 13 of the anterior portion 16 of the lower tray 200, adjacent an anterior lingual surface 204. The anterior surface 401 of the projection 400 thus touches or is adjacent to the anterior lingual surface 204 of the lower tray 200 when the appliance 10 is worn by a user, such that the projection 400 is positioned posteriorly with respect to the anterior portion 16 of the lower tray 200. This positioning limits the anterior movement of the upper dental tray 100 and hence also limits the movement of the upper jaw (maxilla) with respect to the lower dental tray 200 and the lower jaw (mandible) of a user when the present appliance 10 is worn by the user, thereby maintaining the user's jaws in an orientation conducive to preventing or ameliorating sleep apnea and/or snoring.

A proximal (upper) end 402 of the projection 400 connects to an anterior portion of the upper tray 100. Preferably, the projection 400 is in the form of an insert 410 which can be attached to the upper tray 100, such as to the incisal surface 101. The insert can be permanently bonded to, or formed integrally with, the upper dental tray 100, but in a preferred embodiment the projection 400 is made as insert 410, which preferably comprises a base 420 having an upper surface 421, a lower surface 423, an anterior side 426, and a posterior side 428. The base 420 of the insert is sized and shaped to fit into an insert receptacle in the anterior portion of the upper dental tray, preferably located in the occlusal (incisal) surface of the upper dental tray 100. In the illustrated embodiments, the insert receptacle 103 for the base 420 is formed as a slot having an anterior opening 116, an lower surface 111, a right side wall 112, a left side wall 114, and a posterior wall 118. The posterior side 428 of the base 420 is adapted to slide into the slot 103 such that the lower surface 423 of the base 420 faces and/or contacts the lower surface 111 of the slot 103. Preferably, the right side 422 and left side 424 of the base 420 face and/or contact the right side wall 112 and left side wall 114 of the slot 103, respectively. When fully inserted into the slot 103, the posterior side 428 of the base 420 faces and preferably contacts the posterior side (wall) 118 of the slot 103. In one embodiment, the base 420 of the insert is retained in the slot or other form of insert receptacle 103 by an interference fit, i.e. by friction, and is thus reversibly attached to the upper tray 100. In this way, the relative positions of the user's jaws can be adjusted by removing the insert 410 and replacing it with different insert having a different configuration. In other embodiments the insert can be fixed to the slot 103 in ways known to the art.

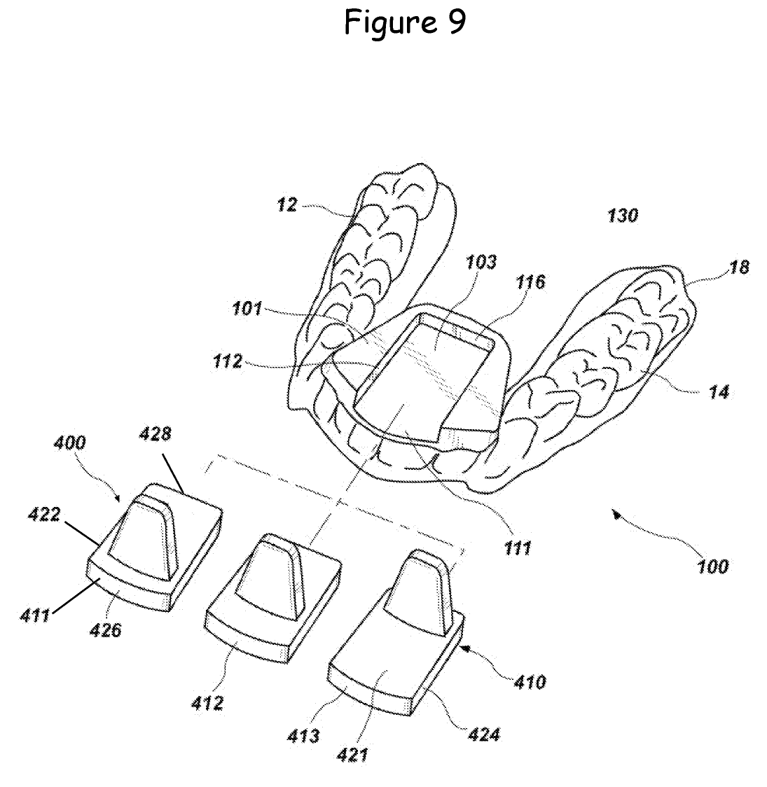

As shown in FIG. 9, inserts 410 can be produced in which the projection 400 is positioned relatively posteriorly or relatively anteriorly. Different patients may require different relative positioning of their upper and lower jaws, and the use of inserts 410 having projections 400 positioned differently allows an upper tray 100 fitted to a user to be produced, after which the correct positioning of the user's jaws can be achieved by using the correct insert 410. FIG. 9, for example, illustrates a series of three inserts 410, namely an anterior insert 411, a medial insert 412, and a posterior insert 413, in which the projection 400 of each is positioned on the base 420 of the insert 410 in a relatively more anterior or posterior position with respect to the projection 400 of the other inserts 410. The projection 400 of anterior insert 411 is positioned most anteriorly, the projection 400 of posterior insert 413 is positioned most posteriorly, and projection 400 of medial insert 412 is positioned between these inserts.

The present appliances 10 can be formed from a variety of orally compatible materials used to make orthodontic appliances, typically polymers such as acrylic. Thermoplastic polymers, thermosets, thermoplastic elastomers, and other materials can also be used. When thermoplastic materials are used, they must be capable of retaining their shape when used by a subject, and thus preferably remain solid at least at about 100° F., and preferably remain solid at somewhat higher temperatures, such as at 110° F., 120° F., or higher. When thermoplastic materials are used to form the present trays, they preferably become deformable at a temperature of 212° F. or less, so that they can be made plastic by being placed in boiling water. Preferably, the material is not deformable at less than 120° F., preferably at not less than 145° F.

### Orthodontic Trays

In one embodiment, the dental trays 15 can be formed as a series of orthodontic dental trays for use by a subject. In this embodiment, a series of upper dental trays 100 and lower dental trays 200 having differently-configured receptacle portions 20 and are applied to the subject over time in order to reposition individual teeth in successive steps and/or to change the configuration of a subject's mandible and/or maxilla. The successive use of a number of such dental trays 15 permits each appliance to be configured to move individual teeth in small increments, typically less than 2 mm, preferably less than 1 mm, and more preferably less than 0.5 mm (referring to the maximum linear translation of any point on a tooth as a result of using a single appliance). The use of the inserts 410 of the present appliance 10 provides a great advantage when orthodontic trays are used in the present invention, because an optimum relative position of the mandible and maxilla of a user can be provided using an insert having a desired length, thereby addressing a user's sleep apnea while allowing orthodonture.

In this embodiment, the tooth-receiving receptacle portions 20 of the dental trays 15 typically have a geometry corresponding to an intermediate or end tooth arrangement intended for a subject. When such a tray 15 is first worn by the subject, certain of the teeth will be misaligned relative to an undeformed geometry of the receptacle portion 20 of a tray 15. In this embodiment, the tray 15 is formed from a material that is sufficiently resilient to accommodate or conform to the misaligned teeth, but will apply sufficient resilient force against such misaligned teeth to reposition the teeth to the intermediate or end arrangement desired for that treatment step. Buttons or other rigid components may be affixed to a subject's teeth, or may be affixed to a portion of one or both of the dental trays 15, in order to apply additional force to a tooth. The appliance will preferably, but not necessarily, fit over all teeth present in the upper or lower jaw. In some cases only certain teeth will be repositioned, and a tray then provides a base or anchor region for holding the repositioning appliance in place as it applies the resilient repositioning force against the tooth or teeth to be repositioned

A subject's teeth are repositioned from an initial tooth arrangement to a final tooth arrangement by placing a series of incremental position adjustment appliances in the subject's mouth. The first tray appliance of the series will have a geometry selected to reposition the teeth from the initial tooth arrangement to a first intermediate arrangement. After the first intermediate arrangement is approached or achieved, one or more additional (intermediate) appliances will be successively placed on the teeth, where such additional appliances have geometries selected to progressively reposition teeth from the first intermediate arrangement through successive intermediate arrangement(s). The treatment will be finished by placing a final appliance in the subject's mouth, where the final appliance has a geometry selected to progressively reposition teeth from the last intermediate arrangement to the final tooth arrangement.

In order to design a series of dental trays 15 that will reposition a particular subject's teeth, a digital data set representing an initial tooth arrangement and a final tooth arrangement can be determined. The initial data set representing the initial tooth arrangement, which can be presented as a visual image, is manipulated to reposition individual teeth. A final digital data set is then produced which represents the final tooth arrangement with repositioned teeth. The initial digital data set may be provided by conventional techniques, including digitizing X-ray images, images produced by computer-aided tomography (CAT scans), images produced by magnetic resonance imaging (MRI), and/or by other methods known to the art for producing three-dimensional digital representations of a subject's teeth. Alternatively, the initial digital data set may be provided by producing a plaster cast of the subject's teeth (prior to treatment) by conventional techniques, for example, and the plaster cast can then be scanned using laser or other scanning equipment to produce a high resolution digital representation of the plaster cast of the subject's teeth.

Once the initial and final data sets have been determined, a series of intermediate data sets, representing intermediate tooth positions for a subject's teeth, are determined. The successive intermediate digital data sets are preferably produced by determining positional differences between selected individual teeth in the initial data set and in the final data set and interpolating the differences. Such interpolation may be performed over at least three discrete stages, embodied in three different dental trays, more often at least ten, sometimes at least twenty-five, and occasionally forty or more. The interpolation can be a linear interpolation for some or all of the positional difference, or alternatively may be nonlinear. The positional differences will correspond to tooth movements where the maximum linear movement of any point on a tooth is preferably 2 mm or less, usually 1 mm or less, and preferably 0.5 mm or less.

Once the intermediate and final data sets have been determined, the appliances can be fabricated, such as with a rapid prototyping device or digital printer. Preferably, the appliance is polymeric and is formed from a thin sheet of a suitable elastomeric polymeric, such as Tru-Tain 0.03 in. thermal forming dental material (Tru-Tain Plastics, Rochester, Minn. 55902). One structure corresponding to each of the dental tray appliances is produced.

The foregoing dental tray appliances and their use in orthodontic treatment are described in U.S. Pat. No. 5,975,893 and in other patents assigned to Align Technology, Inc., including U.S. Pat. Nos. 621,562, 6,217,325, 6,398,548, 6,626,666, 6,629,840, 6,699,037, 7,134,874, 7,474,307, 8,105,080, and 8,562,340. It will be appreciated that dental trays adapted to daytime wear, and including the same tooth-receiving configurations, can be produced so that orthodontic treatment can be pursued both during waking hours and while a user is asleep.

In another embodiment, the present dental trays 15 can be designed to be used in combination with a set of separately formed orthodontic trays 25, typically formed from a polymer material. Each set of such orthodontic trays 25 usually comprises an upper orthodontic tray 300 and a lower orthodontic tray 400. The upper orthodontic tray 300 has an interior surface 311 (for contacting at least some of a subject's teeth), an exterior surface 313, a right side 312, a left side 314, an anterior portion 316, a posterior portion 318, a coronal surface 330, a buccal surface 322, and a lingual surface 324, and the lower orthodontic tray 400 likewise comprises an interior surface, an exterior surface 413, a right side 412, a left side 414, an anterior portion 416, a posterior portion 418, a coronal surface 430, a buccal surface 422, and a lingual surface 424. In this embodiment, the tray receptacle 20 of the present appliance 10 is sized to receive and reversibly retain the orthodontic trays 25, generally by contacting an outer surface of each of the orthodontic trays 25. In this way, a subject can use the orthodontic trays during waking hours and then continue using them at night in combination with the present appliance 10 in order to obtain relief from snoring and/or sleep apnea.

Although the present invention has been described in considerable detail with reference to certain preferred embodiments, other embodiments are possible. The steps disclosed for the present methods, for example, are not intended to be limiting nor are they intended to indicate that each step is necessarily essential to the method, but instead are exemplary steps only. Therefore, the scope of the appended claims should not be limited to the description of preferred embodiments contained in this disclosure.

Recitation of value ranges herein is merely intended to serve as a shorthand method for referring individually to each separate value falling within the range. Unless otherwise indicated herein, each individual value is incorporated into the specification as if it were individually recited herein. All references cited herein are incorporated by reference in their entirety.

1. An oral appliance for treating snoring and/or sleep apnea in a subject, comprising:
(1) an upper dental tray having an anterior portion, a posterior portion, a right side, a left side, a buccal wall, a lingual wall, coronal surface, an interior surface, and an exterior surface, the upper dental tray comprising an incisal surface in the anterior portion and a tray receptacle bounded by the interior surface of the upper dental tray;
(2) a lower dental tray having an anterior portion, a posterior portion, a right side, a left side, a buccal wall, a lingual wall, coronal surface, an interior surface, and an exterior surface, the lower dental tray comprising an incisal surface in the anterior portion and a tray receptacle bounded by the interior surface of the lower dental tray, wherein the incisal surface of the lower dental tray contacts the incisal surface of the upper dental tray when the appliance is worn by the subject; and
(3) a downwardly extending projection having a proximal end, a distal end, an anterior surface, and a posterior surface, wherein the projection is connected to and extends downwardly from the upper dental tray in an anterior portion of the upper dental tray, wherein the distal end of the projection extends below the incisal surface of the lower dental tray such that the anterior surface of the projection contacts or is adjacent to an anterior lingual surface of the lingual wall of the lower dental tray when the appliance is worn by the subject, thereby limiting the anterior movement of the upper dental tray with respect to the lower dental tray and alleviating snoring and/or apnea when the oral appliance is used by the subject,
wherein the receptacles of the upper dental tray and the lower dental tray are each configured to receive and retain an orthodontic tray, or wherein the receptacles of the upper dental tray and the lower dental tray are each configured to reposition one or more teeth of a subject and/or to change the configuration of a subject's mandible and/or maxilla when the appliance is worn by the subject.

2. The oral appliance of claim 1, further comprising a first orthodontic tray and a second orthodontic tray, wherein each of the orthodontic trays comprises an inner surface for contacting at least some of a subject's teeth and an outer surface, and wherein the orthodontic trays can be received within respective receptacles of the upper dental tray and the lower dental tray of the oral appliance such that the outer surface of a respective orthodontic tray contacts the interior surface of a tray receptacle of a respective dental tray.

3. The oral appliance of claim 2, wherein the orthodontic trays comprise a series of first orthodontic trays and a series of second orthodontic trays, and wherein the orthodontic trays in the series comprise tray receptacles having different configurations in order to change the position of the subject's teeth and/or the shape of the subject's jaw.

4. The oral appliance of claim 3, wherein the receptacles of the upper dental tray are shaped to receive all of the first orthodontic trays, and the receptacles of the lower dental tray are shaped to receive all of the second orthodontic trays.

5. The oral appliance of claim 2, wherein the tray receptacles of the dental trays retain the orthodontic trays in a reversibly securable manner.

6. The oral appliance of claim 1, wherein the proximal end of the downwardly extending projection is attached to a base to form an insert, and wherein the anterior portion of the upper dental tray comprises an insert receptacle for receiving the base portion of the insert and reversibly securing the insert to the upper dental tray.

7. The oral appliance of claim 6, wherein the base is reversibly secured to the insert receptacle through an interference fit.

8. The oral appliance of claim 6, wherein the base and the insert receptacle each comprise a right side, a left side, and a posterior side, and wherein the insert receptacle comprises an anterior opening, the base being configured to slide horizontally through the anterior opening such that the right side, left side, and rear side of the base contact the right side, left side, and rear side of the insert receptacle when the insert is positioned in the insert receptacle.

9. The oral appliance of claim 6, further comprising a plurality of inserts, wherein the projection of each of the inserts is positioned on the base of the respective insert in a relatively more anterior or posterior position with respect to the projection of the other inserts.

10. A method of treating sleep apnea and/or snoring, comprising the step of providing the oral appliance of claim 1 to a subject.
